(19) United States
(12) Patent Application Publication
(10) Pub. No.: US 2010/0298709 A1
(43) Pub. Date: Nov. 25, 2010

(54) METHOD FOR NONLINEAR IMAGING OF ULTRASOUND CONTRAST AGENTS AT HIGH FREQUENCIES

(75) Inventors: Andrew Needles, Toronto (CA); James I. Mehi, Thornhill (CA); Desmond Hirson, Thornhill (CA)

Correspondence Address: CLARK & ELBING LLP, 101 FEDERAL STREET, BOSTON, MA, 02110, US

(73) Assignee: VisualSonics Inc., Toronto (CA)

(21) Appl. No.: 12/763,053

(22) Filed: Apr. 19, 2010

Related U.S. Application Data
(60) Provisional application No. 61/170,451, filed on Apr. 17, 2009.

(51) Int. Cl. A61B 8/14

(52) U.S. Cl. 600/458

(57) ABSTRACT

This invention employs multiple ultrasound pulse firings of either alternating phase and/or amplitude to detect nonlinear fundamental and subharmonic signals from microbubble contrast agents within living tissue, at high frequencies (≧15 MHz), e.g., with a linear array transducer. It can be shown that the contrast-to-tissue ratio (CTR) decreases with increasing ultrasound frequency because of nonlinear ultrasound propagation in tissue. However, using the subharmonic signal in addition to the nonlinear fundamental harmonic component, rather than the conventional second harmonic used at lower frequencies, provides appreciable signal strength to overcome the limitations of nonlinear tissue propagation. Additionally, the method provides for the ability to switch, at some desired frequency above 20 MHz, into a purely alternating phase inversion acquisition, in combination with bandpass filtering of the subharmonic frequency band, minimizing the losses in CTR as the frequency increases. This maintains contrast sensitivity for more limited fields of view, as penetration depth will be limited at higher frequencies. Thus, within the same micro-ultrasound imaging system, many applications of microbubble detection can be achieved with a wide range of frequencies that covers both resolution and sensitivity requirements.

### CROSS-REFERENCE TO RELATED APPLICATIONS

This application claims benefit of U.S. Provisional Application No. 61/170,451, filed Apr. 17, 2009, which is hereby incorporated by reference.

### BACKGROUND OF THE INVENTION

The invention relates to the field of nonlinear ultrasound imaging.

Understanding the patterns of blood flow in the microcirculation is a powerful tool for evaluating the differences between normal and pathological tissues. In addition to visualizing and quantifying blood flow in the microcirculation, targeting microbubbles to cellular receptors and detecting them with ultrasound can give valuable insight about the molecular state of small animal models of human disease. Microbubble contrast agents have been used in ultrasound imaging as a means of improving the visualization of blood flow with respect to the surrounding tissue beyond the sensitivity of Power and Color Doppler imaging. These micron-sized particles (˜1-10 μm, on the order of the size of a red blood cell) consist of a gas core surrounded by a lipid shell and are injected in very minute volumes into the circulatory system.

In standard high-frequency (≧15 MHz) B-Mode (gray-scale) imaging, the microbubbles can be visualized because of their high echogenicity from incident ultrasound waves. With post-processing algorithms, these enhanced echoes from bubbles can be segmented from tissue. A disadvantage with this approach, however, is that in many cases the ultrasound echoes from tissue have a comparable magnitude to microbubbles, resulting in poor contrast between the microbubbles and the surrounding tissue. This effect can make visualization of the microbubbles difficult, even after post-processing. Additionally, single-element transducers used for high frequency small animal imaging generally have a fixed focus and narrow depth of field. These characteristics can result in a large variation in acoustic pressure as a function of depth, resulting in large variations in the excitation and detection of microbubbles, which reduces image quality outside of the fixed focus. Single-element transducers must also be mechanically scanned, which limits the ability to send multiple pulse firings down a single image line while maintaining real-time frame rates.

Recent developments in linear array technology have pushed traditional ultrasound imaging frequencies higher, into the range of 15-70 MHz. There exists a need for improved visualization of blood flow at these frequencies.

### SUMMARY OF THE INVENTION

The invention provides ultrasound devices and methods that provide improved sensitivity to microbubble contrast agents. The invention uses multiple transmitted ultrasound pulses, which allows the detection of nonlinear subharmonic frequencies. In certain embodiments, the series of pulses allows for the simultaneous detection of nonlinear fundamental harmonic and subharmonic frequencies.

The invention removes signal originating from surrounding tissue through methods other than post-processing. Eliminating post-processing techniques also allows the visualization of contrast agent to occur in real-time.

In one aspect, the invention features a method for nonlinear ultrasound imaging by transmitting multiple ultrasound pulses having shifted phases or scaled amplitudes or both into a subject and detecting subharmonic signal generated by the microbubble contrast agent. When phase shifting, i.e., inversion, is employed, the method may further include bandpass filtering to detect the subharmonic signal but not nonlinear fundamental signal. When amplitude scaling, e.g., a ratio of 2:1, is employed, the method may further include detecting nonlinear fundamental signal generated by the microbubble contrast agent. Bandpass filtering may also be employed in this process. The method preferable does not detect linear fundamental signal from tissue in the subject and/or second harmonic signal generated by the microbubble contrast agent.

The microbubble contrast agent may be preadministered to the subject or administered as part of the method.

An exemplary center frequency of the transmitted ultrasound is 15 MHz-70 MHz. In certain embodiments, the ultrasound transmitted is defocused by the use of transmit f-numbers of 4 or greater or by the use of a non-standard transmit delay profile to maintain a transmit pressure between 200−500 kPa with depth in tissue.

Detection of echoes from the subject may include quadrature sampling, for example of the form:

$${g}_{Q} ( t ) = {\sum }_{n = - ∞}^{∞} {( - 1 )}^{n} g ( {nT}_{s} ) δ ( t - {nT}_{s} )$$

$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$

where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase.

The method may be employed to image microbubble contrast agents in the vasculature or an organ of the subject is imaged. Exemplary subjects are laboratory animals.

The method may further include obtaining a linear ultrasound image of the subject, and the linear and nonlinear images of the subject may be displayed overlaid or adjacent to one another.

In a related aspect, the invention features an ultrasound system including an arrayed ultrasound transducer; a transmit beamformer capable of generating multiple ultrasound pulses having shifted phases or scaled amplitudes or both; a receive beamformer capable of receiving reflected ultrasound signal from the multiple pulses; a receive filter capable of combining the multiple pulses to determine subharmonic or nonlinear fundamental signal; and a processor capable of producing an ultrasound image from subharmonic or nonlinear fundamental signal.

The system may also be capable of quadrature sampling the received ultrasound signal, wherein the sampling is of the form:

$${g}_{Q} ( t ) = {\sum }_{n = - ∞}^{∞} {( - 1 )}^{n} g ( {nT}_{s} ) δ ( t - {nT}_{s} )$$

$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$

where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase, to produce a sample signal. In other embodiments, the system includes bandpass filters for the detection of the subharmonic and/or nonlinear fundamental signal.

The invention also features a method for quadrature sampling of an ultrasound signal by obtaining an ultrasound signal reflected from a subject; and performing quadrature sampling on the ultrasound signal using a processor, wherein the quadrature sampling is of the form:

$${g}_{Q} ( t ) = {\sum }_{n = - ∞}^{∞} {( - 1 )}^{n} g ( {nT}_{s} ) δ ( t - {nT}_{s} )$$

$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$

where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase, to produce a sample signal. This method may further include generating an ultrasound image from the sampled signal and displaying the ultrasound image.

Preferably, the invention employs a linear array transducer; however, other types of arrayed transducers could be used (e.g., phased, curvilinear phased, or two-dimensional) provided that they do not involve mechanical scanning (e.g., as with an annular array). By using a linear array transducer, the depth-of-field of the ultrasound field can be varied, allowing for optimized excitation and nonlinear detection of contrast agents, with a real-time multi-pulse approach. Additionally, the linear array provides the ability to image contrast agents with multiple pulse firings down a single image line, allowing for multi-pulse signal processing and averaging at high frame rates (>30 Hz).

Other features and advantages will be apparent from the following description and the claims.

### BRIEF DESCRIPTION OF THE DRAWINGS

FIG. 1 is a schematic diagram of functional blocks for an exemplary method of imaging a tissue using ultrasound.

FIGS. 2A-2D are schematic depiction of pulse sequences and the response of tissue and microbubbles. FIG. 2A shows the response of tissue to two pulses that are phase inverted. FIG. 2B shows the response of microbubble contrast agents to two pulses that are phase inverted. FIG. 2C shows the response of tissue to two pulses that have different amplitudes. FIG. 2D shows the response of microbubbles to two pulses that have different amplitudes.

FIG. 3 is a graph showing two-way transducer response for 4 representative elements of a 21 MHz linear array.

FIG. 4 is a graph showing 24 MHz in vitro phase inversion and amplitude scaling frequency spectra for both bubbles and tissue.

FIG. 5 is a graph showing 24 MHz in vitro Contrast-to-Tissue-Ratio (“CTR”).

FIG. 6 is a series of ultrasound images of mouse kidney. The images on the left are linear B-Mode images, and the images on the right are nonlinear images obtained with amplitude scaling.

### DETAILED DESCRIPTION OF THE INVENTION

The invention provides new ultrasound devices and methods for improving sensitivity of microbubble contrast agents.

The subharmonic echo has the unique property that it is generated through nonlinear scattering from microbubbles, but not from tissue. In general, lower frequency contrast agent imaging methods have utilized nonlinear energy at the second harmonic, either exclusively or in addition to nonlinear fundamental signal (U.S. Pat. Nos. 5,577,505 and 6,319,203). This approach is less desirable at higher frequencies for two main reasons. First, the amount of nonlinear tissue signal at the second harmonic, even for relatively low mechanical index (MI) imaging, is significant at high frequencies (Goertz et al., “High frequency nonlinear B-Scan imaging of microbubble contrast agents,” IEEE Trans Ultrason Ferroelectr Freq Cont 2005; 52:65-79). This result suggests that there is a frequency dependence of nonlinear propagation in tissue (Hamilton et al., “Nonlinear acoustics: theory and applications,” Academic Press, 1998). The second reason is that, since ultrasound attenuation increases as a function of frequency, the second harmonic at high frequencies will suffer from more frequency dependent attenuation than at lower frequencies.

Accordingly, the present invention utilizes subharmonic energy instead of second harmonic energy. Goertz et al. (supra) demonstrated that high-frequency subharmonic imaging was possible; however, this approach used a method of analog filtering, which limits the ability to separate linear and nonlinear signal components that overlap in frequency. Using a multi-pulse imaging scheme has the ability to separate overlapping linear and nonlinear frequency components, which in turn allows for larger signal bandwidth. By sending an alternating series of either phase shifted (i.e., inverted) or amplitude scaled pulses (by a factor between 1 and 4), or both, and applying signal processing on the received echoes, nonlinear subharmonic energy from microbubbles, in addition to the nonlinear fundamental energy, can be detected at high transmit frequencies (e.g., 15 MHz and higher).

### Technical Description

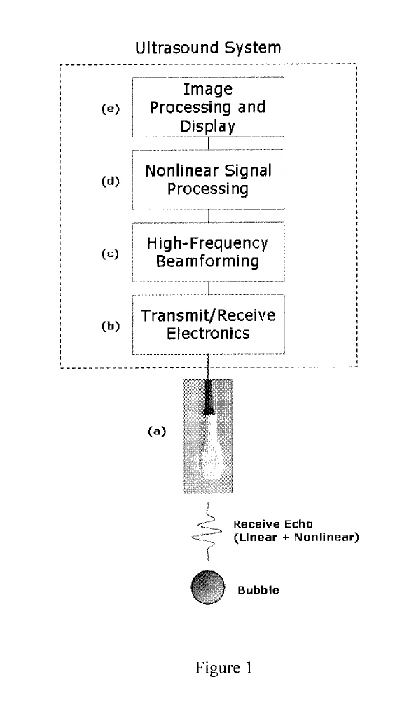

FIG. 1 illustrates functional blocks for the invention. The invention employs a microbubble contrast agent, preferably with a mean diameter between 1-3 μm, a fluorocarbon gas core, and encapsulated with a lipid shell. Other microbubble contrast agents are described herein. This microbubble provides a nonlinear response when excited by high-frequency ultrasound.

Nonlinear imaging can be carried our using any arrayed transducer that does not require mechanical scanning (e.g., linear, phased, curvilinear phased, or two-dimensional).

Ultrasound imaging systems may transmit pulsed energy along a number of different directions, or ultrasonic beams, and thereby receive diagnostic information as a function of both lateral directions across the body and axial distance into the body of a subject. This information may be displayed as two dimensional, “b-scan” images. Such a two-dimensional presentation gives a planar view, or “slice” through the body and shows the location and relative orientation of many features and characteristics within the body. Furthermore, by tilting or moving the ultrasonic sensor across the body, a third dimension may be scanned and displayed over time, thereby providing three-dimensional information. Alternatively, ultrasound returns may be presented in the form of “m-scan” images, where the ultrasound echoes along a particular beam direction are presented sequentially over time, with the two axes being axial distance versus time. Thus, m-scan displays enable diagnosis of rapidly moving structures, such as heart valves. Some ultrasound systems may combine both b-scan and m-scan images within the same display.

Other ultrasound imaging systems may simultaneously present multiple ultrasound information, including b-scan, m-scan and Doppler image displays, along with other information, such as EKG signals and/or phonograms.

Once transmitted the ultrasound interacts with the subject's tissues and the contrast agent. The ultrasound is reflected by structures within the subject and scattered non-linearly by the contrast agent. Echoes resulting from interactions with the subject and contrast agent return to an ultrasound imaging system. After ultrasound is received, it is processed to form an image.

Other ultrasound imaging systems may simultaneously present multiple ultrasound information, including b-scan, m-scan and Doppler image displays, along with other information, such as EKG signals, blood pressure, respiration, and/or phonograms. Image acquisition may be time-registered or triggered using an ECG signal. Alternatively or in addition, image acquisition may be gated or triggered using a respiration waveform.

Preferred embodiments of the invention employ a linear array based ultrasound imaging system. One such system includes a 64 channel, high frequency beamformer, capable of driving linear arrays in the 15-70 MHz range (Foster et al., “A New 15-50 MHz Array-Based Micro-Ultrasound Scanner for Preclinical Imaging,” Ultrasound Med. Biol. 2009; 35:1700-1708; U.S. Patent Application Publication No. 2007/0239001; and PCT Application No. WO2010/033867). Another example of such system includes a 30 MHz, 64-element, 74-micron pitch, linear array design (Lukacs et al., “Performance and characterization of new micromachined high-frequency linear arrays,” IEEE Trans Ultrason Ferroelec Freq Contr 2006; 53:1719-1729). Yet another example of such as system is a 256-element high-frequency (40-MHz) linear array using a high-frequency 1-3 PZT-polymer composite material (Brown et al., “Fabrication and performance of a 40-MHz linear array based on a 1-3 composite with geometric elevation focusing,” IEEE Trans Ultrason Ferroelectr Freq Cont 2007; 54:1888-1894).

A linear array brings the advantage of improved depth-of-field, a critical parameter for microbubble contrast agent excitation and detection. Additionally, a linear array provides the ability to image contrast agents with multiple pulse firings down a single image line, allowing for multi-pulse signal processing and averaging at high frame rates (>30 Hz). The number of pulse firings down a single line ranges from a minimum of 2, to as many 8 or more, although imaging frame rate will decrease with increasing pulse firings.

### (a) High-Frequency Linear Array

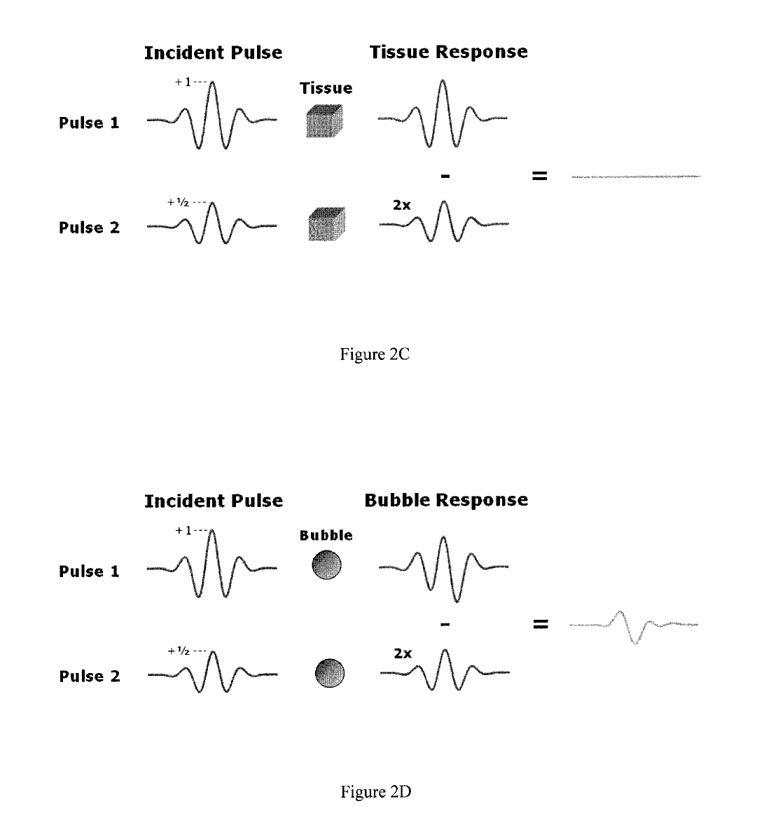

A preferred high-frequency linear array has a minimum of 256 elements, with an approximate two-way bandwidth of at least 70%, and a center frequency of at least 15 MHz. FIG. 3 is a frequency plot of a two-way transducer response for 4 representative elements of a 21 MHz array (MS-250, VisualSonics).

### (b) Transmit/Receive Electronics

The high-frequency transmitter preferably generates square-wave pulse trains for exciting the array elements, e.g., in the range of approximately 3-40 volts peak. On the Vevo 2100 imaging system, the transmitter employs two voltage sources, VP1 and VP2. These voltages can be set to arbitrary values, with the restriction that VP2<=VP1. In the case of amplitude scaling specifically, two voltage sources are required; phase inversion only uses VP1. With the particular implementation of the transmit electronics on the Vevo™ 2100, the supply voltages VP1 and VP2 are not necessarily the voltages that ultimately excite the transducer elements. The voltages that excite the elements are typically slightly lower (˜0.5-1 V) than the supply voltage because of voltage drops in the transmit electronics. The voltage that ultimately excites the transducer elements, however, will be converted to an acoustic pressure for transmission into the imaging subject. The ratio of these acoustic pressures is known so that the received echoes from different amplitude transmissions can be properly compensated and cancelled. Any residual signal after applying the cancelation is taken to be the contrast agent signal.

By varying supply voltages and measuring the actual voltages, V1 and V2, across a given array element, a linear relationship is observed. By performing linear regression, the following equations can be obtained:

V1=A₁·VP1+B₁

and

V2=A₂·VP2+B₂

where A₁, A₂, B₁, and B₂, are the coefficients obtained from the linear regression. By setting βₜᵣₐₙₛₘᵢₜ=V1/V2 and solving the system of two equations above for VP2, the following empirical relationship can be expressed:

$$VP 2 = \frac{[ \frac{( {A}_{1} \cdot VP 1 + {B}_{1} )}{{β}_{transmit}} - {B}_{2} ]}{{A}_{2}} ,$$

where βₜᵣₐₙₛₘᵢₜ is the expected ratio between VP1/VP2. The value of βₜᵣₐₙₛₘᵢₜ is predetermined, and the supply voltages are set to account for any voltage drop errors in the transmit circuitry, such that the ratio of the actual voltages applied to the transducer elements satisfy the predetermined ratio of βₜᵣₐₙₛₘᵢₜ. Typically, βₜᵣₐₙₛₘᵢₜ=2 when performing amplitude scaling. When βₜᵣₐₙₛₘᵢₜ=2, typical values are A₁=1.351, A₂=1.305, B₁=0.085, B₂=−1.205, and the actual VP2 can be calculated based on a VP1 that has been set. Alternatively, the coefficients may be selected so as to result in the lowest level of tissue clutter in the fundamental frequency band after processing.

Methods of phase shifting or amplitude scaling for other specific ultrasound systems will be known to the user.

The receive electronics detect small received signals, on the order of millivolts, while maintaining low electronic noise.

### (c) High-Frequency Beamforming

The beamformer provides the combined processes of transmitting and receiving ultrasound signals, e.g., on a maximum of 64 elements of the linear array simultaneously, and with variable delays on individual channels. Additionally, upon reception of the ultrasound echoes, beamforming includes the digital sampling process and summation of individual channels.

The ability to vary the number and timing between individual elements (channels) of the array is a key component for preferred embodiments of the invention. By defocusing the transmit ultrasound beam (e.g., using a higher f-number, where f-number is defined as the ratio of focal depth to aperture size, or by using a non-standard delay profile where not all elements are focused to the same depth), a more consistent transmit pressure (e.g., 200-500 kPa) can be maintained with depth in tissue, ensuring that all microbubbles are responding in a similar fashion, thus maximizing sensitivity to the contrast agent. Defocusing may be achieved by using a smaller number of elements to generate a transmit beam for a given depth. In the limiting case, only a single element would be used for transmission, and the beam would be defocused to the point that it was based on the diffraction limit of the single element aperture. Typically, this case would not be applied, but it illustrates the effect of using a smaller number of elements. A non-standard delay profile could be achieved by using sub-groups of elements within an aperture that create a focal point at different depths. The overall effect would be a transmit beam that was a superposition of the beams created by the sub-groups of elements. Transmit pressure is directly proportional to the transmit voltage used to excite the transducer elements. On the Vevo™ 2100, the minimum transmit voltage is 3V which, depending on the location of the transmit focus and path length attenuation, may correspond to a transmit pressure higher than 500 kPa. By using a higher f-number for the transmit beam, the transmit pressure can be reduced even further once the lower limit of the transmit voltage is reached, which minimizes tissue nonlinearities. A typical transmit f-number to be used for the purpose of reducing transmit pressure and improving depth of field is 4 to 16. This f-number results in a loss of lateral resolution in the transmit beam; however, much of this is regained by dynamically keeping the receive beam in focus with the receive portion of the beamformer. The lateral resolution of the imaging system will be a function of both transmit and receive beams. By dynamically varying the aperture size and signal delays on individual channels during reception of the ultrasound echoes prior to summation, the receive beamforming process can keep the receive ultrasound beam in focus throughout the entire image depth. Since the overall two-way resolution of the system is a function of transmit and receives beams, it will be dominated by the smaller beamwidth, in this case the receive beam.

The invention may also utilize a novel baseband quadrature sampling scheme that doubles the bandwidth of current quadrature sampling techniques. This scheme allows for the detection of harmonic components (namely the subharmonic) from contrast agents, as well as wideband fundamental signal. It also ensures that any unwanted second harmonic signal from tissue is sampled properly and does not fold back into the frequency range of interest through aliasing. The sampling process allows unaliased quadrature sampling of signals having a spectrum in the range 0 to fₛ. It requires that in-phase and quadrature samples be acquired at a rate of fₛ, with the delay of Tₛ/2 (where Tₛ=1/fₛ) between quadrature and in-phase samples, and that alternating quadrature sample pairs are multiplied by −1. The sampling process is described algebraically below:

$${g}_{Q} ( t ) = {\sum }_{n = - ∞}^{∞} {( - 1 )}^{n} g ( {nT}_{s} ) δ ( t - {nT}_{s} )$$

$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$

where n is the discrete time variable, Tₛ is the sampling period, δ(t) is the delta function, g(t) is the received ultrasound signal from both tissue and microbubbles, and g_{Q}(t) and g_{I}(t) are the quadrature and in-phase sampled portions of this signal respectively, which are 90° out of phase. This same sampling method is applied for generating ultrasound images of tissue (B-Mode).

### (d) Nonlinear Signal Processing

In the described method, an even numbered length series of two or more pulses is transmitted with either alternating phase and/or amplitude. This alternation can be expressed in general by a factor β_{receive}, which must be applied to every other received echo, g_{N}(t), in the series. Receive filtering is performed on the received echoes to extract the nonlinear signal from microbubbles, y(t). The receive filter scales the echoes to compensate for amplitude scaling or phase shifting of the transmitted ultrasound. For the two pulse case (N=2), the receive filter is:

$$y ( t ) = \frac{{g}_{1} ( t ) + {β}_{receive} \cdot {g}_{2} ( t )}{2}$$

In terms of the sampled components, the receive filter is:

$${y}_{Q} ( t ) = \frac{{g}_{Q 1} ( t ) + {β}_{receive} \cdot {g}_{Q 2} ( t )}{2} {y}_{I} ( t ) = \frac{{g}_{I 1} ( t ) + {β}_{receive} \cdot {g}_{I 2} ( t )}{2}$$

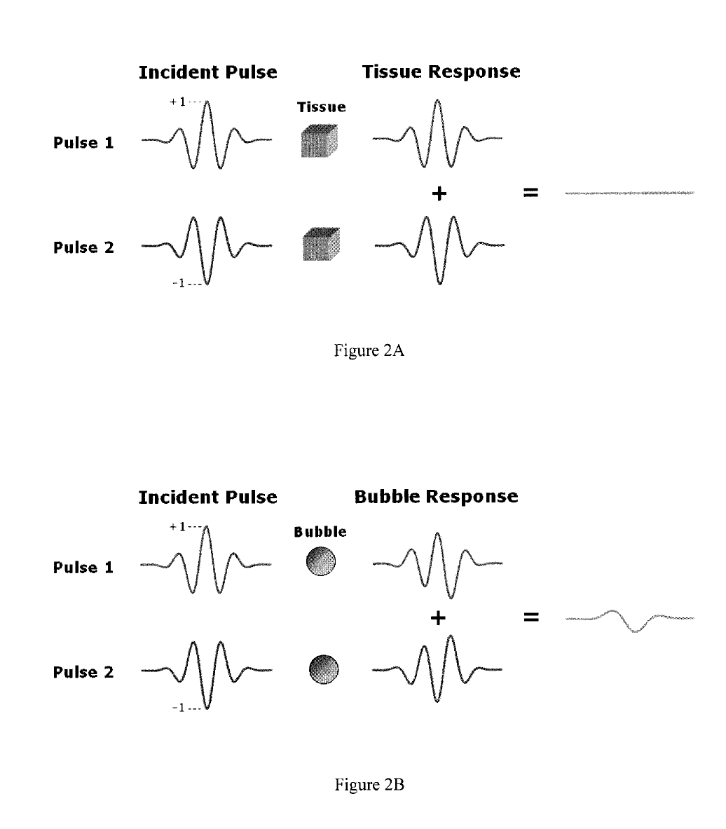

For the case where the second transmit pulse is one half the amplitude of the first transmit pulse, β_{receive} equals −2. For the case where the second transmit pulse is phase inverted relative to the first transmit pulse, β_{receive} equals 1. For the case where the second transmit pulse is phase inverted and amplitude scaled by one half, β_{receive} equals 2. Exemplary transmitted and received pulses for tissue and microbubbles are shown schematically in FIGS. 2A-2D. In FIG. 2A, two phase inverted pulses are transmitted into a subject, and the echoes received from tissue are combined to result in no signal. In FIG. 2B, two phases inverted pulses are transmitted into a subject, and the echoes received from the microbubbles are combined to produce a detectable signal. In FIG. 2C, two amplitude scaled pulses are transmitted into a subject, and the echoes received from tissue are combined to result in no signal. In FIG. 2B, two amplitude scaled pulses are transmitted into a subject, and the echoes received from the microbubbles are combined to produce a detectable signal.

More generally, the method applies the following receive filter to an even numbered length series of more than two pulses:

$$y ( t ) = \frac{\begin{matrix}\frac{1}{2} {g}_{1} ( t ) + {β}_{receive} \cdot {g}_{2} ( t ) + {g}_{3} ( t ) + … + \\ {g}_{N - 1} + \frac{1}{2} {β}_{receive} \cdot {g}_{N} ( t )\end{matrix}}{N}$$

And again, in terms of the sampled components, the receive filter is:

$${y}_{Q} ( t ) = \frac{\begin{matrix}\frac{1}{2} {g}_{Q 1} ( t ) + {β}_{receive} \cdot {g}_{Q 2} ( t ) + {g}_{Q 3} ( t ) + … + \\ {g}_{QN - 1} + \frac{1}{2} {β}_{receive} \cdot {g}_{QN} ( t )\end{matrix}}{N}$$

$${y}_{I} ( t ) = \frac{\begin{matrix}\frac{1}{2} {g}_{I 1} ( t ) + {β}_{receive} \cdot {g}_{I 2} ( t ) + {g}_{I 3} ( t ) + … + \\ {g}_{IN - 1} + \frac{1}{2} {β}_{receive} \cdot {g}_{IN} ( t )\end{matrix}}{N}$$

This pulse sequence includes pairs of a standard pulse with a phase shifted and/or amplitude scaled pulse. In certain embodiments, the modulation of the standard pulse is the same in each pair of the sequence. In other embodiment, the modulation of the standard pulse can differ among pairs in the sequence. The output of the receive filter, y(t), may then be bandpass filtered about the appropriate frequency band, depending on the application. Typical frequency cutoffs for the bandpass filter are centered on the fundamental frequency, the subharmonic frequency, or both. The bandwidth of the filter cutoff should be set according to the bandwidth of the transmitted ultrasound pulses used to form the contrast image. This typically corresponds to a fractional −6 dB two-way bandwidth (relative to the center frequency) ranging from 50-100%. Standard ultrasound image processing techniques are then applied to the filtered y(t) signal. This includes envelope detection, log compression, scan conversion, and display mapping.

### (e) Image Processing and Display

Conventional ultrasound image processing strategies may be applied, as described in Becher et al., “Handbook of contrast echocardiography,” Berlin: Springer, 2000. For example, spatial filters are applied within the image frame, and persistence algorithms are applied between image frames. These algorithms enhance image quality and tissue delineation. Alternatively, the invention may employ quantitative analysis of the raw microbubble echo power as a function of time.

Other image processing and display strategies include a system for producing an ultrasound image using line-based image reconstruction with the contrast agents and the methods provided herein. One example of such a system may have the components as described in PCT Publication No. WO 2010/033867, U.S. Pat. No. 7,052,460, and U.S. Patent Application Publication No. 2004/0236219, which are incorporated herein by reference. Ultrasound images are formed by the analysis and amalgamation of multiple pulse echo events. An image is formed, effectively, by scanning regions within a desired imaging area using individual pulse echo events, referred to as “A-Scans,” or ultrasound lines. Each pulse echo event requires a minimum time for the acoustic energy to propagate into the subject and to return to the transducer. The image is completed by “covering” the desired image area with a sufficient number of scan lines, referred to as “painting in” the desired imaging area so that sufficient detail of the subject anatomy can be displayed. The number of and order in which the lines are acquired can be controlled by the ultrasound system, which also converts the raw data acquired into an image. Using a combination of hardware electronics and software instructions in a process called “scan conversion,” or image construction, the ultrasound image obtained is rendered so that a user viewing the display can view the subject being imaged. When imaging contrast agents, interleaved image frames of tissue (B-Mode) and contrast agent can be displayed beside one another simultaneously, or with the contrast agent image overlaid on top of the B-Mode image.

### Contrast Agents

Examples of commercial microbubble contrast agents include, but are not limited to, MicroMarker™, Definity®, Sonovue™, Levovist™ and Optison®. Examples of microbubble contrast agents are described in U.S. Pat. Nos. 5,529,766; 5,558,094; 5,573,751; 5,527,521; 5,547,656; 5,769,080; 5,552,782; 5,425,366; 5,141,738; 4,681,119; 4,466,442; 4,276,885; 6,200,548; 5,911,972; 5,711,933; 5,686,060; 5,310,540; and 5,271,928. Other suitable contrast agents are described in WO2005/070472, which is incorporated herein by reference.

A typical contrast agent comprises a thin flexible or rigid shell composed of albumin, lipid or polymer confining a gas such as nitrogen or a perfluorocarbon. Other examples of representative gases include air, oxygen, carbon dioxide, hydrogen, nitrous oxide, inert gases, sulfur fluorides, hydrocarbons, and halogenated hydrocarbons.

Liposomes or other microbubbles can also be designed to encapsulate gas or a substance capable of forming gas as described in U.S. Pat. No. 5,316,771. In another embodiment, gas or a composition capable of producing gas can be trapped in a virus, bacteria, or cell to form a microbubble contrast agent.

A contrast agent can be modified to achieve a desired volume percentage by a filtering process, such as by micro or nano-filtration using a porous membrane. Contrast agents can also be modified by allowing larger bubbles to separate in solution relative to smaller bubbles. For example, contrast agents can be modified by allowing larger bubbles to float higher in solution relative to smaller bubbles. A population of microbubbles of an appropriate size to achieve a desired volume percentage can subsequently be selected. Other methods are available in the art for separating micron-sized and nano-sized particles and can be adapted to select a microbubble population of the desired volume of submicron bubbles such as by centrifugation, such as methods described in WO2005/070472, which is incorporated herein by reference. For optical decorrelation methods, a Malvern™ Zetasizer or similar apparatus may be used.

Suitable microbubble contrast agents also include targeted contrast agents. Several strategies can be used to direct ultrasound contrast agent to a desired target. One strategy takes advantage of the inherent chemical properties of the microbubble shell components. For example, albumin or lipid microbubbles can attach to the surface of target cells via cell receptors. Another strategy involves conjugation of specific ligands or antibodies that bind to desired markers.

A targeted contrast agent is an ultrasound contrast agent that can bind selectively or specifically to a desired target. Such selective or specific binding can be readily determined using the methods and devices described herein. For example, selective or specific binding can be determined in vivo or in vitro by administering a targeted contrast agent and detecting an increase in non-linear ultrasound scattering from the contrast agent bound to a desired target. Thus, a targeted contrast agent can be compared to a control contrast agent having all the components of the targeted contrast agent except the targeting ligand. By detecting increased non-linear resonance or scattering from the targeted contrast agent versus a control contrast agent, the specificity or selectivity of binding can be determined. If an antibody or similar targeting mechanism is used, selective or specific binding to a target can be determined based on standard antigen/epitope/antibody complementary binding relationships.

Further, other controls can be used. For example, the specific or selective targeting of the microbubbles can be determined by exposing targeted microbubbles to a control tissue, which includes all the components of the test tissue except for the desired target ligand or epitope. To compare a control sample to a test sample, levels of non-linear resonance can be detected by enhanced ultrasound imaging.

Specific or selective targeted contrast agents can be produced by methods known in the art, for example, using the methods described. For example, targeted contrast agents can be prepared as perfluorocarbon or other gas-filled microbubbles with a monoclonal antibody on the shell as a ligand for binding to target ligand in a subject as described in Villanueva et al., “Microbubbles Targeted to Intracellular Adhesion Molecule-1 Bind to Activated Coronary Artery Endothelial Cells,” Circulation 1998; 98: 1-5. For example, perfluorobutane can be dispersed by sonication in an aqueous medium containing phosphatidylcholine, a surfactant, and a phospholipid derivative containing a carboxyl group. The perfluorobutane is encapsulated during sonication by a lipid shell. The carboxylic groups are exposed to an aqueous environment and used for covalent attachment of antibodies to the microbubbles by the following steps. First, unbound lipid dispersed in the aqueous phase is separated from the gas-filled microbubbles by floatation. Second, carboxylic groups on the microbubble shell are activated with 1-ethyl-3-(3-dimethylaminopropyl) carbodiimide, and antibody is then covalently attached via its primary amino groups with the formation of amide bonds.

Targeted microbubbles can also be prepared with a biotinylated shell as described in Weller et al., “Modulating Targeted Adhesion of an Ultrasound Contrast Agent to Dysfunctional Endothelium,” Ann. Biomed. Engineering 2002; 30:1012-1019. For example, lipid-based perfluorocarbon-filled microbubbles can be prepared with monoclonal antibody on the shell using avidin-biotin bridging chemistry using the following protocol. Perfluorobutane is dispersed by sonication in aqueous saline containing phosphatidyl choline, polyethylene glycol (PEG) stearate, and a biotinylated derivative of phosphatidylethanolamine as described in the art. The sonication results in the formation of perfluorobutane microbubbles coated with a lipid monolayer shell and carrying the biotin label. Antibody conjugation to the shell is achieved via avidin-biotin bridging chemistry. Samples of biotinylated microbubbles are washed in phosphate-buffered saline (PBS) by centrifugation to remove the lipid not incorporated in the microbubble shell. Next, the microbubbles are incubated in a solution (0.1-10 μg/mL) of streptavidin in PBS. Excess streptavidin is removed by washing with PBS. The microbubbles are then incubated in a solution of biotinylated monoclonal antibody in PBS and washed again. The resultant microbubble have antibody conjugated to the lipid shell via biotin-streptavidin-biotin linkage. In another example, for targeted microbubbles, biotinylated microbubbles can be prepared by sonication of an aqueous dispersion of decafluorobutane gas, distearoylphosphatidylcholine, polyethyleneglycol-(PEG-)stearate, and distearoyl-phosphatidylethanolamine-PEG-biotin. Microbubbles can then be combined with streptavidin, washed, and combined with biotinylated echistatin.

Targeted microbubbles can also be prepared with an avidinated shell, as is known in the art. In a preferred embodiment, a polymer microbubble can be prepared with an avidinated or streptavidinated shell. For example, a polymer contrast agent comprising a functionalized polyalkylcyanoacrylate can be used, as described in patent application PCT/EP01/02802. Streptavidin can be bonded to the contrast agent via the functional groups of the functionalized polyalkylcyanoacrylate. In a preferred embodiment, avidinated microbubbles can be used in the methods disclosed herein.

When using avidinated microbubbles, a biotinylated antibody or fragment thereof or another biotinylated targeting molecule or fragments thereof can be administered to a subject. For example, a biotinylated targeting ligand such as an antibody, protein or other bioconjugate can be used. Thus, a biotinylated antibody, targeting ligand or molecule, or fragment thereof can bind to a desired target within a subject. Once bound to the desired target, the contrast agent with an avidinated shell can bind to the biotinylated antibody, targeting molecule, or fragment thereof. When bound in this way, high frequency ultrasound energy can be transmitted to the bound contrast agent, which can produce non-linear scattering of the transmitted ultrasound energy. An avidinated contrast agent can also be bound to a biotinylated antibody, targeting ligand or molecule, or fragment thereof prior to administration to the subject.

When using a targeted contrast agent with a biotinylated shell or an avidinated shell a targeting ligand or molecule can be administered to the subject. For example, a biotinylated targeting ligand such as an antibody, protein or other bioconjugate, can be administered to a subject and allowed to accumulate at a target site. A fragment of the targeting ligand or molecule can also be used.

When a targeted contrast agent with a biotinylated shell is used, an avidin linker molecule, which attaches to the biotinylated targeting ligand can be administered to the subject. Then, a targeted contrast agent with a biotinylated shell is administered to the subject. The targeted contrast agent binds to the avidin linker molecule, which is bound to the biotinylated targeting ligand, which is itself bound to the desired target. In this way a three step method can be used to target contrast agents to a desired target. The intermediate targeting ligand can bind to all of the desired targets detailed above as would be clear to one skilled in the art.

Targeted contrast agents or non-targeted contrast agents can also comprise a variety of markers, detectable moieties, or labels. Thus, a microbubble contrast agent equipped with a targeting ligand or antibody-incorporated into the shell of the microbubble can also include another detectable moiety or label. As used herein, the term “detectable moiety” is intended to mean any suitable label, including, but not limited to, enzymes, fluorophores, biotin, chromophores, radioisotopes, colored particles, electrochemical, chemical-modifying or chemiluminescent moieties.

As with non-targeted contrast agents, targeted contrast agents can be modified to achieve a desired volume percentage for high frequency imaging by a filtering process, such as by micro or nano-filtration using porous membranes. Sizing of the microbubbles can occur before or after the microbubbles are adapted to be targeted. For example, a desired size microbubble population can be selected prior to implementing the protocols detailed above for producing a targeted microbubble contrast agent.

Thus, for both targeted and non-targeted ultrasound contrast agents, a desired percentage by volume of microbubbles can be selected to enhance ultrasound imaging by non-linear scattering of the contrast agent and thus to enhance ultrasound imaging. Such a population can be selected as described above, by being compared to a control population have all of the components of the test sample of microbubbles except for a difference in microbubble size.

Administration of contrast imaging agents of the present invention may be carried out in various fashions, such as intravascularly, intralymphatically, parenterally, subcutaneously, intramuscularly, intraperitoneally, interstitially, hyperbarically, orally, or intratumorly using a variety of dosage forms. One preferred route of administration is intravascularly. For intravascular use the contrast agent is generally injected intravenously, but may be injected intraarterially as well. The useful dosage to be administered and the mode of administration may vary depending upon the age and weight of the subject, and on the particular application intended. Typically, dosage is initiated at lower levels and increased until the desired contrast enhancement is achieved. Generally, the contrast agent construed in accordance with embodiments of the invention is administered in the form of an aqueous suspension such as in water or a saline solution (e.g., phosphate buffered saline). The water can be sterile and the saline solution can be a hypertonic saline solution (e.g., about 0.3 to about 0.5% NaCl), although, if desired, the saline solution may be isotonic. The solution also may be buffered, if desired, to provide a pH range of pH 6.8 to pH 7.4. In addition, dextrose may be included in the media.

The contrast agent can be administered intravenously to a laboratory animal. A laboratory animal includes, but is not limited to, a rodent such as a mouse or a rat. As used herein, the term laboratory animal is also used interchangeably with small animal, small laboratory animal, or subject, which includes mice, rats, cats, dogs, fish, rabbits, guinea pigs, rodents, etc. The term laboratory animal does not denote a particular age or sex. Thus, adult and newborn animals, as well as fetuses (including embryos), whether male or female, are included.

In one embodiment, the contrast agent is administered intravenously to a mouse or a rat. In another embodiment, the contrast agent is administered into the tail vein of a mouse or a rat. The intravenous injection can be administered as a single bolus dose, or by repeated injection or continuous infusion. Effective dosages and schedules for administering the compositions may be determined empirically, and making such determinations is within the ordinary skill in the art. The dosage range for the administration of the compositions are those large enough to produce the desired ultrasound imaging effect. Such an effect typically includes an increased return from the contrast agent versus a reduced return from surrounding tissue. The dosage should not be so large as to cause adverse side effects. Generally, the dosage will vary with the ultrasound imaging protocol and the desired imaging characteristics, and can be determined by one skilled in the art. The dosage can be adjusted by the individual researcher. Dosage can vary, and can be administered in one or more dose administrations daily, for one or several days. The ultrasound can be transmitted immediately after administration of contrast agent or at any time interval subsequent to contrast agent administration. Ultrasound imaging can also begin prior to administration, continue throughout the administration process, and continue subsequent to the completion of administration. The imaging can also take place at any discrete time prior to, during or after administration of the contrast agent.

### Uses

The invention can be used to image the vasculature of a subject (e.g., a human or non-human mammal (such as a mouse, rat, guinea pig, or rabbit). The methods above can also be used to image organs of a laboratory animal. The organs imaged can include, but are not limited to a lung, heart, brain, kidney, liver, and blood. In one embodiment, the organ imaged is the organ of a mouse or a rat. The compositions and methods can also be used to image physiological or pathological processes such as angiogenesis or a neoplastic condition in a laboratory animal.

Other conventional uses for ultrasound imaging may be applied, as described in Lindner, “Molecular imaging with contrast ultrasound and targeted microbubbles,” J Nucl Cardiol 2004; 11:215-221. Other uses include molecular imaging of targets using target contrast agents, imaging of angiogenesis, and other medical imaging applications (Lyshchik et al., “Molecular imaging of vascular endothelial growth factor receptor 2 expression using targeted contrast-enhanced high-frequency ultrasonography,” J Ultrasound Med 2007; 26:1575-1586; Ritter et al., “A 30 MHz piezo-composite ultrasound array for medical imaging applications,” IEEE Ultrason Ferroelec Freq Cont 2002; 49:217-230; Rychak et al., “Microultrasound molecular imaging of vascular endothelial growth factor receptor 2 in a mouse model of tumor angiogenesis,” Mol Imaging 2007; 6:289-296).

The methods described herein include embodiments wherein the contrast agent is disrupted or destroyed by a pulse of ultrasound. The pulse of ultrasound can be produced by the same or a different transducer as the transducer producing the imaging frequency ultrasound. Therefore, the above methods contemplate using a plurality of ultrasound probes and frequencies.

Other possible uses of the methods include imaging liquid filled sub-micron sized particles which have inherently poor echogenicity to ultrasound. By exposing the particles to a high powered ultrasound pulse they can be vaporized and converted to a gas bubble (Kripfgans et al., Ultrasound Med. Biol., 26:1177-1189, 2000). The resulting gas bubble can be imaged with the methods described here, with improved contrast and sensitivity relative to the liquid particle. As these liquid particles are much smaller than microbubble contrast agents, they can escape from the vascular space, prior to being vaporized and imaged. This technique allows the particles to be targeted to cellular receptors other than those found on vascular endothelial cells prior to being vaporized and imaged.

### EXAMPLES

### Example 1

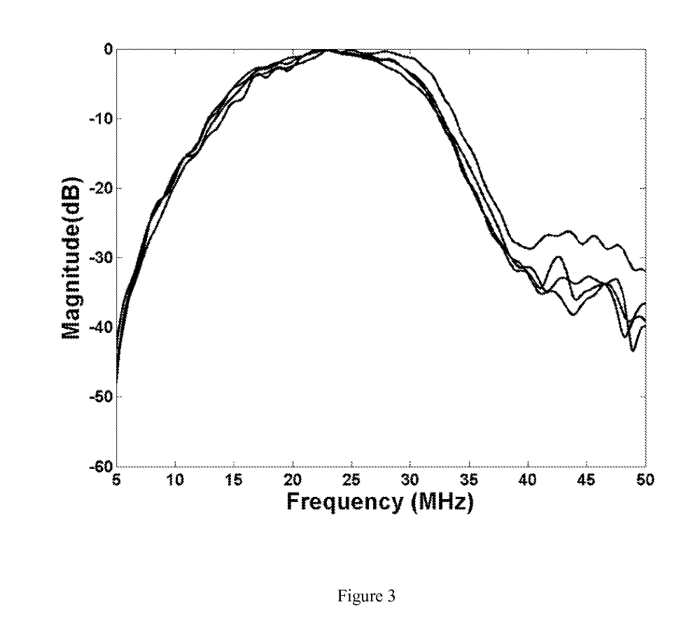

FIG. 4 shows data collected with a 21 MHz linear array (MS-250, VisualSonics, Toronto) at a transmit frequency of 24 MHz. The array was connected to a VisualSonics Vevo 2100 micro-ultrasound imaging system. The system is capable of beamforming 64 channels of data. The resulting summation from the 64 channels can be recorded digitally in baseband quadrature format and offloaded from the system for processing and analysis. The data are from MicroMarker (VisualSonics, Toronto) high frequency contrast agent flowing through a tissue-mimicking medium, using either phase inversion or amplitude scaling. FIG. 3 is a frequency plot of received ultrasound echoes, with all curves referenced to the raw unprocessed data (not shown). As shown in FIG. 4, both phase inversion and amplitude scaling detect nonlinear subharmonic energy at 12 MHz. In the case of amplitude scaling, additional nonlinear energy is detected at the fundamental frequency (24 MHz). In addition, phase inversion is better at suppressing signal from tissue, particularly in the fundamental band. The residual tissue signal at the fundamental frequency detected by amplitude scaling is nonlinear in nature, despite the fact that the data were collected at a relatively low acoustic pressure (350 kPa).

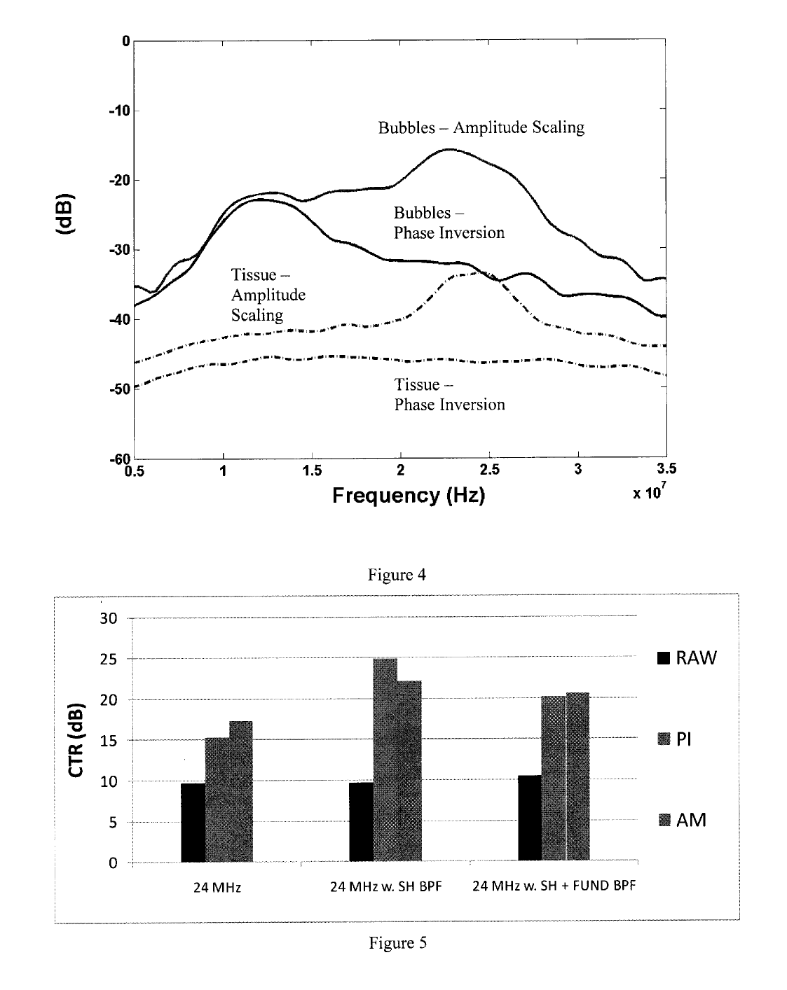

By applying bandpass filtering (BPF) to the data shown in FIG. 4, energy in specific frequency bands can be isolated. Such filtering increases sensitivity, with the trade-off that bandwidth (i.e., axial resolution) is reduced. FIG. 5 summarizes the results of the 24 MHz data in terms of Contrast-to-Tissue-Ratio (CTR), with different bandpass filters. By applying a bandpass filter, the CTR can be increased above that of the raw unprocessed data. Furthermore, FIG. 4 demonstrates that it is possible to take advantage of the improved tissue suppression offered by phase inversion (PI) by applying a bandpass filter around the subharmonic signal only (SH BPF). Such tissue suppression is desirable as transmit frequencies are increased (e.g., to 30 MHz and above), and nonlinear tissue signal becomes more prevalent. For intermediate frequencies, e.g., 15-30 MHz, amplitude scaling (AM) can be applied to detect both subharmonic and nonlinear fundamental signal (SH+FUND BFP) with higher spatial resolution in the axial dimension than that obtained with pulse inversion.

### Example 2

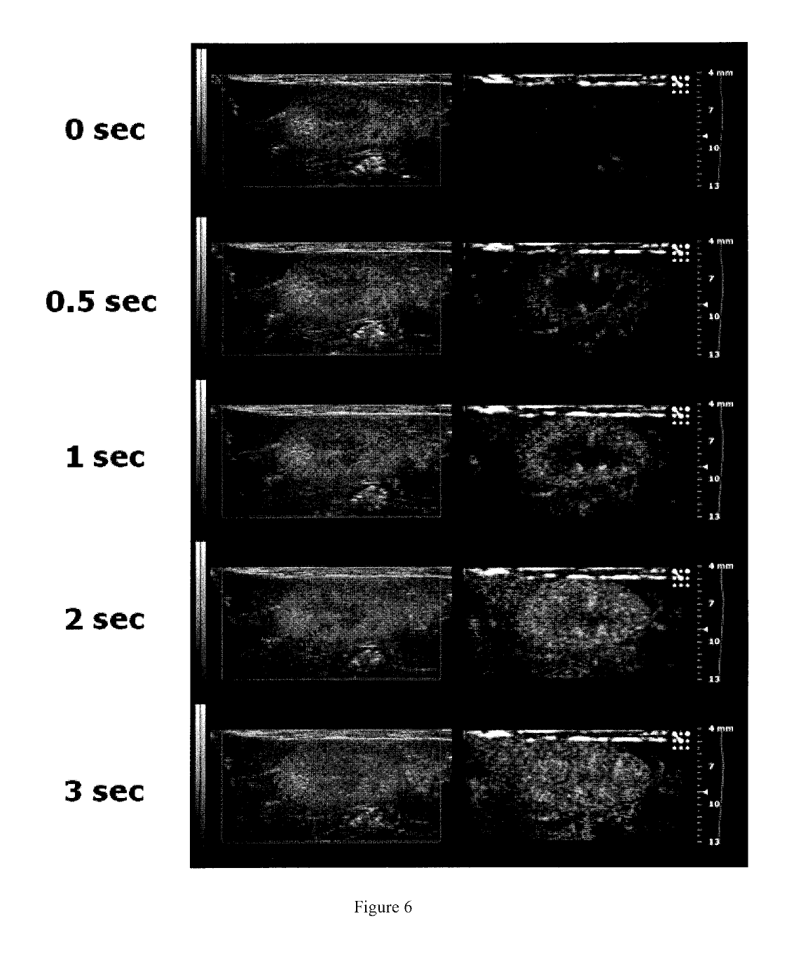

An adult female mouse was administered a single 50-μl bolus of MicroMarker contrast agent (1.2·×10⁷ bubbles per bolus) and imaged with amplitude scaling at 18 MHz using a Vevo 2100 ultrasound imagining platform (VisualSonics). The nonlinear contrast agent signal (right) is shown simultaneously with B-Mode images (left) in FIG. 6. The sequence of images shows the contrast enhancement attributable to the bolus over time. The scan plane was oriented from the dorsal side of the mouse, through a long section of the kidney.

### Other Embodiments

Throughout this application, various publications are referenced. The disclosures of these publications in their entireties are hereby incorporated by reference.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Other embodiments are in the claims.

What is claimed is:

1. A method for nonlinear ultrasound imaging, said methods comprising (i) transmitting multiple ultrasound pulses having shifted phases or scaled amplitudes or both into a subject; (ii) detecting subharmonic signal generated by the microbubble contrast agent, thereby imagining the subject nonlinearly.

2. The method of claim 1, wherein the ultrasound pulses in step (i) have shifted phases.

3. The method of claim 2, further comprising employing bandpass filtering to detect the subharmonic signal but not nonlinear fundamental signal.

4. The method of claim 1, wherein the ultrasound pulses in step (i) have scaled amplitudes.

5. The method of claim 1, wherein step (ii) further comprises detecting nonlinear fundamental signal generated by the microbubble contrast agent.

6. The method of claim 5, wherein linear fundamental signal from tissue in the subject is not detected.

7. The method of claim 5, further comprising applying bandpass filtering to detect the subharmonic and nonlinear fundamental signal.

8. The method of claim 1, wherein second harmonic signal generated by the microbubble contrast agent is not detected in step (ii).

9. The method of claim 1, wherein the microbubble contrast agent is preadministered to the subject.

10. The method of claim 1, further comprising, prior to step (i), administering the microbubble contrast agent to the subject.

11. The method of claim 1, wherein the center frequency of the transmitted ultrasound is 15 MHz-70 MHz.

12. The method of claim 1, wherein the ultrasound transmitted in step (i) is defocused by the use of transmit f-numbers of 4 or greater or by the use of a non-standard transmit delay profile to maintain a transmit pressure between 200-500 kPa with depth in tissue.

13. The method of claim 1, wherein steps (i) and (ii) employ a linear array transducer.

14. The method of claim 1, wherein step (ii) comprises quadrature sampling.

15. The method of claim 14, wherein the quadrature sampling is of the form:
$${g}_{Q} ( t ) = {\sum }_{n = - ∞}^{∞} {( - 1 )}^{n} g ( {nT}_{s} ) δ ( t - {nT}_{s} )$$
$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$
where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase.

16. The method of claim 1, wherein the microbubble contrast agents in the vasculature or an organ of the subject are imaged.

17. The method of claim 1, wherein the subject is a laboratory animal.

18. The method of claim 1, further comprising obtaining a linear ultrasound image of the subject.

19. The method of claim 18, wherein the linear and nonlinear images of the subject are displayed overlaid or adjacent to one another.

20. A method for quadrature sampling of an ultrasound signal, said method comprising the steps of:
(i) obtaining an ultrasound signal reflected from a subject; and
(ii) performing quadrature sampling on the ultrasound signal using a processor, wherein the quadrature sampling is of the form:
$${g}_{Q} ( t ) = {\sum }_{n = - ∞}^{∞} {( - 1 )}^{n} g ( {nT}_{s} ) δ ( t - {nT}_{s} )$$
$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$
where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase, to produce a sample signal.

21. The method of claim 20, further comprising generating an ultrasound image from the sampled signal.

22. The method of claim 21, further comprising displaying the ultrasound image.

23. An ultrasound system comprising:
(i) an arrayed ultrasound transducer;
(ii) a transmit beamformer capable of generating multiple ultrasound pulses having shifted phases or scaled amplitudes or both;
(iii) a receive beamformer capable of receiving reflected ultrasound signal from the multiple pulses;
(iv) a receive filter capable of combining the multiple pulses to determine subharmonic or nonlinear fundamental signal; and
(iv) a processor capable of producing an ultrasound image from subharmonic or nonlinear fundamental signal.

24. The ultrasound system of claim 23, wherein the system is capable of quadrature sampling the received ultrasound signal, wherein the sampling is of the form:
$${g}_{Q} ( t ) = {\sum }_{n = - ∞}^{∞} {( - 1 )}^{n} g ( {nT}_{s} ) δ ( t - {nT}_{s} )$$
$${g}_{I} ( t ) = {\sum }_{n = - ∞}^{∞} {( - 1 )}^{n} g ( {nT}_{s} + \frac{{T}_{s}}{2} ) δ ( t - {nT}_{s} - \frac{{T}_{s}}{2} )$$
where n is the discrete time variable, Tₛ is the sampling period, δ(t) is the delta function, g is the received ultrasound signal from the subject, and g_{Q2} and g_{I2} are the quadrature and in-phase sampled portions of this signal respectively, which are 90° out of phase, to produce a sample signal.
